(19) United States
(12) Patent Application Publication
(10) Pub. No.: US 2009/0068166 A1
(43) Pub. Date: Mar. 12, 2009

(54) METHOD AND COMPOSITION FOR PROTECTION AGAINST RADIATION

(76) Inventor: Ning Man Cheng, Hong Kong (CN)

Correspondence Address: EAGLE IP LIMITED, 22/F., KWAI HUNG HOLDINGS CENTRE, 89 KING'S ROAD, NORTH POINT, HK

(21) Appl. No.: 12/282,647

(22) PCT Filed: Mar. 15, 2007
(86) PCT No.: PCT/IB2007/050888

§ 371 Date: Sep. 11, 2008

Related U.S. Application Data
(60) Provisional application No. 60/783,040, filed on Mar. 17, 2006.

(51) Int. Cl. A61K 38/46; C12N 9/14; A61P 35/04; A61P 43/00

(52) U.S. Cl. 424/94.6; 435/195

(57) ABSTRACT

Methods are described for using an arginine depleting agent such as arginase and derivatives thereof, which reduce physiological arginine levels, as radioprotectants to protect normal mammalian cells from DNA damage caused by ionizing radiation. Treatment can result in the protection of normal tissues in cancer patients undergoing radiotherapy and in protection from the hazardous effects of exposure to radiological dispersal devices or occupational and environmental ionizing radiation.

### FIELD OF INVENTION

This invention relates to the use of arginase and other arginine depleting agents for the protection of normal cells from radiation damage and in particular for the reduction of symptoms during and after radiation therapy for treatment of malignancies.

### BACKGROUND OF INVENTION

Radiation therapy uses high-energy rays or particles to kill cancer cells. Radiation therapy works because ionizing radiation destroys the cancer cells' ability to replicate. Patients can be treated with radiation therapy alone, or in combination with other cancer treatment modalities, such as chemotherapy and/or surgery.

Radiation therapy damages normal tissues surrounding the tumor and this leads to many undesirable side effects. These side effects are more profound in the radiation treatment of esophageal cancer, head and neck cancers, lung cancer and colorectal cancer where there are lots of collateral damage to surrounding tissues. In most incidences, side effects of radiation therapy become apparent about two weeks into the treatment, when mucositis (inflammation of the mucus membrane of the mouth or bowel mucosa), loss of taste sensation, dry mouth and skin reactions set in. Mucositis is one of the main side effects that render radiation intolerable for some patients. Very often patients lose weight because of failure to ingest and take fluid. In extreme cases, a gastrostomy tube may need to be inserted to maintain adequate nutrition. There is also the risk of swallowing muscles atrophy and permanent swallowing problems as a late sequalae.

Not infrequently, xerostomia (dryness of mouth) becomes a permanent disability even after the cancer is cured. In the case of radiation to rectal tumors, side effects include enteritis and proctitis, causing diarrhea and bleeding which may be acute or chronic. Both of which are extremely difficult to treat.

### SUMMARY OF INVENTION

In light of the foregoing background, it is an object of the present invention to provide protection to normal tissues during the course of radiation therapy so as to minimize the collateral adverse side effects of radiation.

The mechanism by which radiation causes damage to normal tissue is by ionization of atoms in the cells. More specifically, in the nucleus where genetic materials or DNAs are stored. Ionizing radiation in radiation therapy induces DNA damage. If a radiation damaged cell needs to perform a function it has to have time to repair itself before it can perform a specific function. Radiation damaged cells may die if such repair does not take place. During S phase, cells start DNA synthesis in which the chromosomes are copied. Radiation damage to DNA often takes place during the S phase. Cells damaged by radiation are more readily repaired while they are in G₁ and G₀ phase. There are two cell cycling check-points in G1 and G2 phase. These safeguard genomic stability and ensure DNA stability and functional integrity. These check-points also prevent cells from entering mitosis or M phase if the DNA are damaged during the phase of last division, providing an opportunity for DNA repair and stopping the proliferation of damaged cells.

An important aspect of the present invention is the recognition that within the normal body, conditions that would prevent the division of normal cells for a prescribed amount of time would protect these cells from radiation damage during this prescribed period.

Accordingly, the present invention, in one aspect, is a method of protecting normal cells from radiation damage by administering an effective dose of an arginine depleting agent to a patient for a prescribe period preferably before any anticipated damage to the normal cell's DNA, for example, before radiation therapy. The prescribed period may be, for example, at least one day. Other examples are 1-7 days, 1-5 days and most preferably 1-3 days.

A preferred embodiment of the present invention is the use of arginase for the protection of normal cells during radiation therapy, whereby in a certain preferred embodiment, the radiation therapy is for the treatment of human malignancies. Arginine depleting agents may be arginase of various types, arginase, arginine deiminase, or any other biological or chemical compounds that may be administered to the human body for the depletion of arginine. In another preferred embodiment, the arginine depleting agent is human recombinant arginase I. In another preferred embodiment, the human recombinant arginase I is modified to increase its half live to at least one day. In yet another preferred embodiment, the recombinant arginase I is pegylated to increase its serum half life to at least one day. In the most preferred embodiment, the pegylated arginase has an increased serum half life of at least three days. One way of making such human recombinant arginase I is disclosed in WO 04/000349 A1.

A further aspect of the invention is the use of arginase in the manufacture of a medicament for the protection of normal cells during radiation therapy. Such radiation therapy is, in a preferred embodiment, for the treatment of human malignancies.

Yet another aspect of the invention is the use of arginase for protection from the DNA damaging effects of radiation contamination, such as from ‘dirty bombs’.

### BRIEF DESCRIPTION OF FIGURES

FIG. 1 is a chart of the cell cycle distribution of Hep3B cells treated with arginase-SPA-PEG5,000 for 3 days, at concentrations of 0 to 0.5 IU/ml.

FIG. 2 is a chart of the cell cycle distribution of Hep3B cells treated with arginase-SPA-PEG5,000 for 3 days, at concentrations of 1 to 50 IU/ml.

FIG. 3 is a chart of the cell cycle distribution of Hep3B cells treated with arginase-SPA-PEG5,000 for 3 days, at concentrations of 100 IU/ml.

FIG. 4 is a chart of the cell cycle distribution of Alexander Cell Line (PLC/PRF/5) cells treated with arginase-SPA-PEG5,000 for 3 days, at concentrations of 0 to 0.5 IU/ml.

FIG. 5 is a chart of the cell cycle distribution of PLC/PRF/5 cells treated with arginase-SPA-PEG5,000 for 3 days, at concentrations of 1 to 50 IU/ml.

FIG. 6 is a chart of the cell cycle distribution of PLC/PRF/5 cells treated with arginase-SPA-PEG5,000 for 3 days, at concentrations of 100 IU/ml.

FIG. 7 is a bar chart of the cell cycle distribution of human foreskin fibroblasts (HFF-1) cells after pegylated recombinant human arginase I and/or 5FU treatment.

### DETAILED DESCRIPTION

As used herein and in the claims, “comprising” means including the following elements but not excluding others. The term “dirty bomb(s)” refers to a radiological dispersal device or radiation contamination, for example, a bomb that leaves considerable radioactive contamination, an atom bomb, or a combination thereof. The term “depleting,” as used herein and in the claims, is defined as the removal of arginine to such extent so as to trigger cell cycle arrest in normal cells, such extent can be determined by a person of ordinary skill in the art.

Each time radiation therapy is given it involves a balance between destroying the cancer cells and sparing the normal cells. Radiation damage to normal tissues during radiation therapy often causes late skin injury, carcinogenesis, leukemogenesis, and genetic damage from the ionizing radiation. The degree of radiation damage correlates with the degree of active cell division. For example, actively dividing cells of the bone marrow, mucus membrane and hair follicles will be subject to more damage. The overall damage to tissues is also proportional to the duration for which ionizing radiation is applied, due to accumulation of ions in the cells.

The general side effects of radiation therapy are lethargy and malaise. There may also be varying degrees of site specify injury to skin, soft tissues, muscles, nerves, secretary glands, and the gastrointestinal tract, wherever radiation is administered. The cell cycle is important in cancer treatment because radiation is more effective on cells that are actively dividing. It is less effective on cells that are in the resting phase (G₀).

The present invention teaches using arginine depletion to protect normal cells during or after radiation exposure. Arginase depletes arginine as described by the present inventor in WO2004/000349 A1, the details of which are incorporated herein in their entirety. The inventors recognize that arginase acts to deplete arginine, thereby resulting in intracellular accumulation of uncharged arg-tRNA, which, in turn, arrests protein synthesis for example through the shut-off of wortmannin and/or rapamycin sensitive signaling sequence in cells. In an example, an effective dose of arginase is administered to a patient during the radiation therapy for treatment of head and neck cancers including nasopharyngeal cancer, whereby radiation damage to the normal cells of the nasopharynx and oral cavity are markedly reduced.

The following examples are experiments that demonstrated the differing effects of pegylated recombinant human arginase I on a normal cell line and a malignant cell line. All references cited are incorporated in their entirety herein.

### EXAMPLE ONE

### Effectiveness of Pegylated Recombinant Human Arginase I on HFF-1 Normal Cell after 1 and 3 Day Incubation

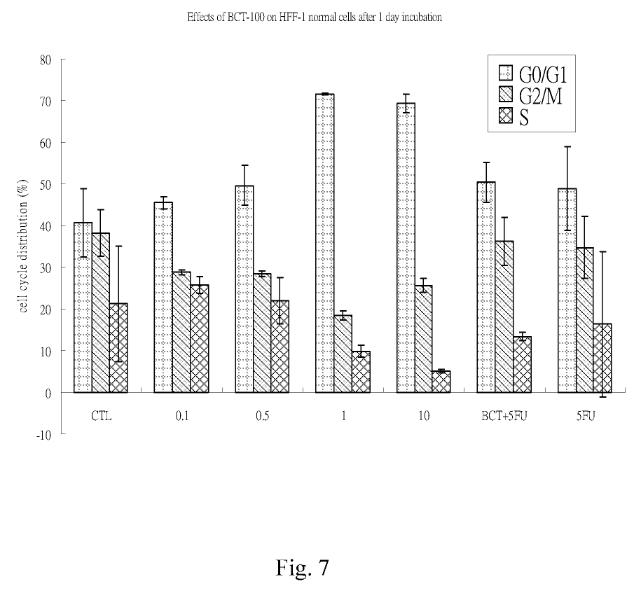

The human foreskin fibroblasts (HFF-1) normal cell line was seeded in low cell density of 5×10⁴ cells/well onto 6-well plate and grown for 1 day before addition of pegylated recombinant human arginase I. The plates were incubated with pegylated recombinant human arginase I at concentrations of 0.1, 0.5, 1, 5, 10, and 50 U/ml, respectively. 10 U/ml pegylated recombinant human arginase 1 and 10 μg/ml of 5-fluorouracil (5-FU) were also added as a combination treatment, and 10 μg/ml of 5-FU was added as a control. Then cells were incubated at 37° C., 5% CO_{2/95}% air incubator for 1 day and 3 days. After incubation with the relevant treatment, cells were trypsinized and fixed with 70% ethanol for at least 30 minute in the dark at −20° C. Fixed cells were then washed with PBS twice and stained with propidium iodide (PI) staining solution (10 μl 2 mg/ml PI and 50 μl 10 mg/ml RNase A in 400 μl PBS) for at least 30 minutes at 37° C. The stage within the cell cycle of each cell was analyzed by flow cytometer (using BD FACSDiva flow cytometer and ModFit software). The results are shown in Table 1, Table 2 and FIG. 7.

It can be seen that at 1 U/ml concentrations of pegylated recombinant human arginase I after 1 day incubation, more HFF-1 cells were stopped at G0/G1 after arginine depletion when compared to our untreated cells. Normal somatic cells will start their cellular repair mechanism during G0/G1 phase after sustaining DNA damage. Hence this data show that DNA damage that may be sustained due to radiation can be reduced in normal cells if they have been previously treated by pegylated recombinant human arginase I.

TABLE 1
Effects of pegylated recombinant human arginase
I on HFF-1 normal cell after 1 day incubation

| | G0/G1 | G2/M | S |
|---|---|---|---|
| Control | 40.635 | 38.135 | 21.23 |
| 0.1 | 45.5 | 28.815 | 25.68 |
| 0.5 | 49.635 | 28.38 | 21.99 |
| 1 | 71.595 | 18.445 | 9.86 |
| 10 | 69.335 | 25.62 | 5.045 |
| BCT + 5FU | 50.38 | 36.225 | 13.395 |
| 5FU | 48.825 | 34.765 | 16.41 |

TABLE 2
Effects of pegylated recombinant human arginase
I on HFF-1 normal cell after 3 day incubation

| | G0/G1 | G2/M | S |
|---|---|---|---|
| Control | 74.97 | 8.49 | 16.54 |
| 0.1 | 76.89 | 11.045 | 12.065 |
| 0.5 | 76.065 | 14.875 | 9.06 |
| 1 | 70.365 | 10.075 | 19.56 |
| 5 | 57.405 | 20.66 | 21.94 |
| 10 | 49.565 | 21.91 | 28.525 |
| BCT + 5FU | 60.78 | 17.16 | 22.07 |
| 5FU | 66.86 | 25.65 | 7.49 |

### EXAMPLE TWO

### Effectiveness of Arginase on Protection of Normal Cells From Damage During Radiation Treatment for Liver Cancer

The cell line used for in vitro testing was Hep3B.

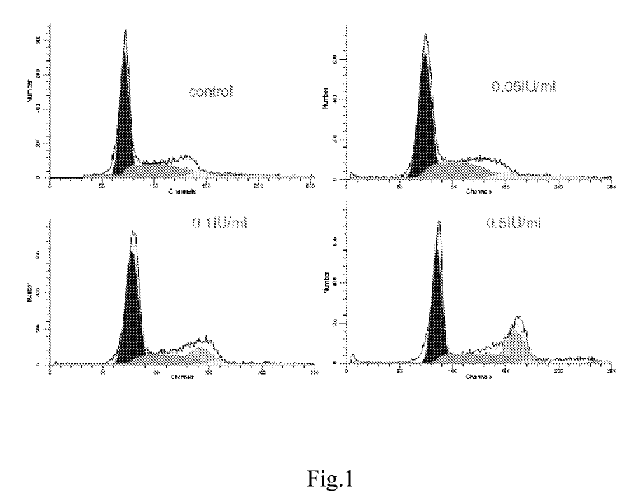

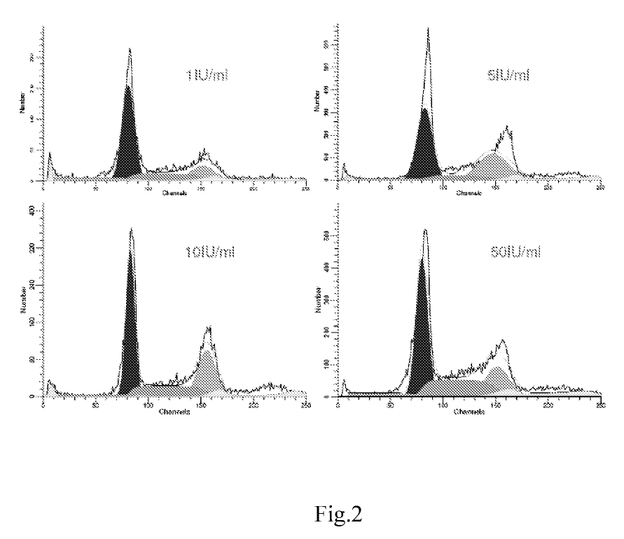

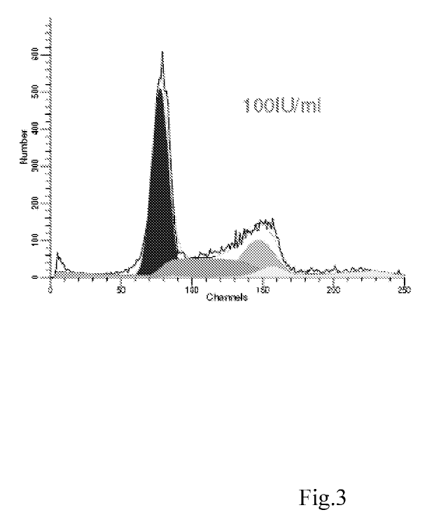

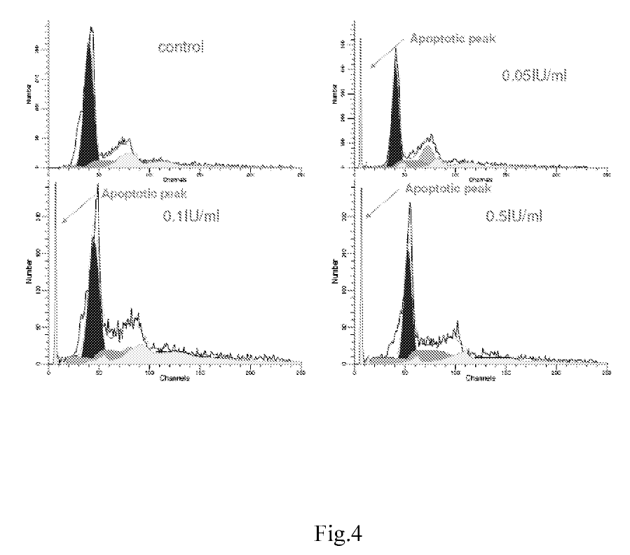

The Hep3B cell line was incubated with Arginase-SPA-PEG-5,000 at concentrations of 0, 0.05, 0.1, 0.5, 1, 5, 10, 50, and 100 IU/ml, respectively, for duration of 3 days. The structure of Arginase-SPA-PEG-5,000 is shown in FIG. 1. The cell distribution of Hep3B after incubation is shown in FIG. 2-4. The results are shown in Table 3 (Table 3 is the flow cytometric data on the effects of arginine deprivation on cell cycle phase distribution in heptocellular carcinoma (HCC) cell line Hep3B). This data shows that cancer cells such as Hep3B are sensitive to arginine deprivation at the appropriate arginase concentrations.

TABLE 3
Summary results of flow cytometry. At higher concentrations
of pegylated recombinant human arginase I, more Hep3B entered
G2/M phase despite low cytosolic levels of arginine.

| Arginase-SPA- | | | |
|---|---|---|---|
| PEG-5,000 (IU/ml) | G0/G1 (%) | S (%) | G2/M (%) |
| 0 | 57.96 | 32.34 | 9.89 |
| 0.05 | 59.93 | 30.87 | 9.71 |
| 0.1 | 58.19 | 24.77 | 17.58 |
| 0.5 | 50.67 | 19.71 | 24.88 |
| 1 | 48.42 | 24.18 | 21.94 |
| 5 | 44.15 | 19.97 | 28.26 |
| 10 | 45.57 | 25.89 | 30.43 |
| 50 | 44.83 | 33.67 | 22.72 |
| 100 | 41.16 | 34.66 | 24.04 |

### EXAMPLE THREE

### Effectiveness of Arginase on Protection of Normal Cells From Damage During Radiation Treatment for Liver Cancer (Using Cancer Cell Line Alexander Cell Line (PLC/PRF/5))

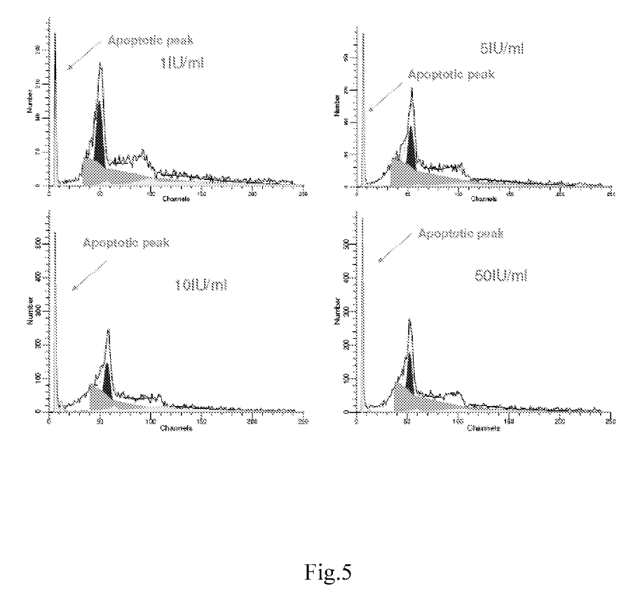

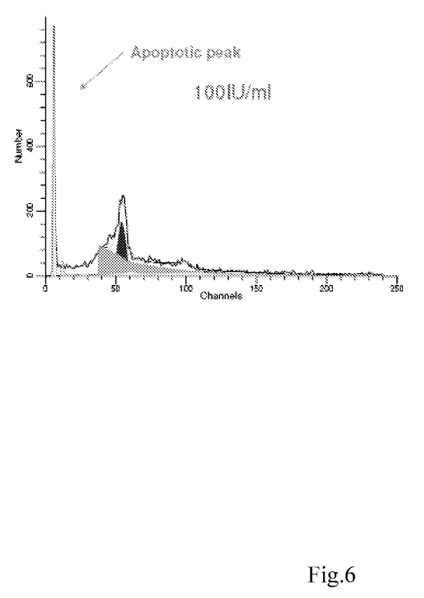

The PLC/PRF/5 cell line was incubated with Arginase-SPA-PEG-5,000 at concentrations of 0, 0.05, 0.1, 0.5, 1, 5, 10, 50, and 100 IU/ml, respectively, for duration of 3 days. The same materials and methods as in Example 1 were used in this example. The cell distribution of PLC/PRF/5 after incubation is shown in FIG. 5-7. The results are shown in Table 4. Thus, it was shown that at higher concentrations of pegylated recombinant human arginase I, more PLC/PRF/5 cells entered S phase, but failed to enter G₂/M. At levels of >50 IU/ml, some PLC/PRF/5 cells died of apoptotic death.

TABLE 4
Summary results from flow cytometry. At higher concentrations
of pegylated recombinant human arginase I, more PLC/PRF/5 cells
entered S phase, but failed to enter G2/M. At levels of >50
IU/m, some PLC/PRF/5 cells died of apoptotic death.

| Arginase-SPA- | | | | |
|---|---|---|---|---|
| PEG-5,000 (IU/ml) | Sub-G1 (%) | G0/G1 (%) | S (%) | G2/M (%) |
| 0 | 0.12 | 71.74 | 15.51 | 12.75 |
| 0.05 | 3.49 | 60.48 | 15.09 | 17.86 |
| 0.1 | 4.33 | 60.92 | 22.74 | 16.32 |
| 0.5 | 6.38 | 59.70 | 29.22 | 11.08 |
| 1 | 6.06 | 57.95 | 23.21 | 16.20 |
| 5 | 8.47 | 59.61 | 20.37 | 20.07 |
| 10 | 10.37 | 58.42 | 20.91 | 15.78 |
| 50 | 19.53 | 55.46 | 24.19 | 14.17 |
| 100 | 27.81 | 51.53 | 36.13 | 6.15 |

Comparing Examples 1, 2 and 3, it can be seen that HCC cell death in relation to pegylated recombinant human arginase I is cell line dependent. In Hep3B cells, there is G0/G1 arrest at low concentrations and G2/M arrest at higher concentrations of pegylated recombinant human arginase I. However, in PLC/PRF/5 cells, pegylated recombinant human arginase I mainly causes S phase arrest and at high concentrations also apoptosis. G1 phase is where the cell makes proteins in preparation for cell division. This phase normally lasts for 18 to 30 hours. The G0 phase is a resting stage where cells have not yet started to divide, only when the cell is signaled to reproduce does it move into the G1 phase. It has thus been shown that most malignant cells, such as the Hep3B cells and PLC/PRF/5 cells have defective “R” checkpoints and are thus committed to cell cycling irrespective of the absence or low concentration of arginine. Malignant cells are therefore more susceptible to radiation damage. As seen from Example 1, normal somatic cells stop cell division at the G1 phase. Hence, it was shown that normal somatic cells are not actively dividing after treatment with pegylated recombinant human arginase I in the absence or low concentration of arginine and DNA damage, e.g. due to radiation, can be minimized.

Furthermore, it appears that the depletion of arginine has a dose-dependent effect on cancer cells that is more than mere depletion of an amino acid. It could be that arginine depletion results in further activation of a defective cell-cycling pathway in cancer cells. Thus, the administration of an arginine depleting enzyme to a patient before and/or during radiation treatment may not only protect the patient from radiation damage, but also have synergistic effect on the killing of the cancer cells.

The preferred embodiments of the present invention are thus fully described. Although the description referred to particular embodiments, it will be clear to one skilled in the art that the present invention may be practiced with variation of these specific details. Hence this invention should not be construed as limited to the embodiments set forth herein. For example, the present invention may also be useful for protection against radiation contamination, for example, ‘dirty bombs’ designed to cause damage to DNA and cells.

1. A method of protecting normal cells from DNA damage by administering an effective dose of an arginine depleting agent to a patient for a prescribed period of time.

2. The method of claim 1, wherein said DNA damage is caused by radiation therapy.

3. The method of claim 2, wherein said radiation therapy is for the treatment of human malignancies.

4. The method of claim 1, wherein said DNA damage is caused by a “dirty bomb” or other environmental ionizing radiation.

5. The method of claim 1, wherein said arginine depleting agent is human arginase I, human arginase II or arginine deiminase.

6. The method of claim 1, wherein administration of said arginine depleting agent is before and/or during radiation therapy.

7. A method of protecting normal cells from DNA damage during radiation therapy comprising the following sequential or simultaneous steps:
a) administering an arginine depleting agent; and
b) administering said radiation therapy.

8. A method of treating human malignancies comprising the steps of:
a) administering an effective dose of an arginine depleting agent for a prescribed period; and
b) administering radiation therapy to treat said malignancies.

9. The method according to claim 1 wherein said arginine depleting agent is recombinant human arginase I or II having a serum half-life of at least 3 days.

10. Use of arginase in the manufacture of a medicament for the protection of normal cells from DNA damage.

11. The use of claim 10, wherein said DNA damage is caused by radiation therapy.

12. The use of claim 11, wherein said radiation therapy is for the treatment of human malignancies.

13. The use of claim 10, wherein said DNA damage is caused by a “dirty bomb” or other environmental ionizing radiation.

14. Use of arginase in the manufacture of a medicament for the treatment of malignancies in combination with radiation therapy.

15. The use according to claim 10 wherein said arginase is recombinant human arginase I or II having a serum half-life of at least 3 days.

16. The method according to claim 7 wherein said arginine depleting agent is recombinant human arginase I or II having a serum half-life of at least 3 days.

17. The method according to claim 8 wherein said arginine depleting agent is recombinant human arginase I or II having a serum half-life of at least 3 days.

18. The use according to claim 14 wherein said arginase is recombinant human arginase I or II having a serum half-life of at least 3 days.
